(19) United States
(12) Patent Application Publication
(10) Pub. No.: US 2011/0112369 A1
(43) Pub. Date: May 12, 2011

(54) ENDOSCOPE SET

(75) Inventors: Lothar Sterling, Waldkirch (DE); Stefan Schlenker, Freiburg (DE); Karl Maechner, Staufen (DE); Hartmund Biedermann, Denzlingen (DE)

(73) Assignee: Scholly Fiberoptic GmbH, Denzlingen (DE)

(21) Appl. No.: 12/915,435

(22) Filed: Oct. 29, 2010

(30) Foreign Application Priority Data
Nov. 11, 2009 (DE) 102009052524.6-35

(51) Int. Cl. A61B 1/07; A61B 1/06

(52) U.S. Cl. 600/178; 600/182

(57) ABSTRACT

An endoscope (1) in which at least two light outlets (7, 10) for illuminating the field of view are provided, each of which forms a light cone (8, 11). The angles of beam spread (9, 12) of the light cones (8, 11) are different and at least the light cone (11) with the smaller angle of beam spread (12) can be switched on and off.

### CROSS-REFERENCE TO RELATED APPLICATIONS

This application claims the benefit of German Patent application 102009052524.6-35, filed Nov. 11, 2009, which is incorporated herein by reference as if fully set forth.

### BACKGROUND

The invention relates to an endoscope set with an endoscope, for example, for bronchoscopy, at least one first light source and at least one first optical waveguide, wherein the first optical waveguide is attached to the first light source and is connected at the distal end of the endoscope to a first light outlet, wherein the first light outlet is designed for illuminating the field of view of the endoscope with light of the first light source in a first light cone with a first angle of beam spread.

Endoscope sets for bronchoscopy and for other medical and special non-medical applications are provided at their distal end with a wide-angle objective. For illuminating the field of view, the endoscope sets are equipped with a wide-angle fiber that generates, at its light outlet, a light cone with the widest possible spread. Due to the wide spread that should provide a field of view that is illuminated as equally as possible, the range of the light cone is limited. Therefore, at a certain range or depth from the distal end, the endoscope set can provide no more illumination to the objective. In order to be able to perform the diagnosis or biopsy in the bronchoscopy more efficiently and more reliably, however, many times it is typical to take a brief look into deeper ramifications of the bronchia. For this purpose, the distal end must be inserted into these ramifications. This method causes a considerable time delay in the examination.

### SUMMARY

The invention is based on the desire to provide an endoscope, in particular, for bronchoscopy, in which the time required for the examination of ramified internal spaces is as small as possible.

To address this issue, in the case of an endoscope of the type described above, it is provided that, on the distal end of the endoscope, a second light outlet is provided, wherein this second light outlet is designed for illuminating the field of view of the endoscope with light in a second light cone with a second angle of beam spread, wherein the second angle of beam spread is smaller than the first angle of beam spread, and an activation device is provided with which the second light cone can be turned on. In this way, turning on the second light cone is to be understood as a change in the operating mode of the endoscope set according to which the intensity of the emitted light within the second light cone is greater after the change than before the change. The invention thus offers the advantage that the surgeon or user of the endoscope set can turn on the second light cone as a far-reaching light beam when needed, in order to take a look at structures farther away, without the increased light intensity blinding and interfering with the rest of the examination. Guiding the objective to the structure that is farther away to examine the same is thus no longer necessary and the time required for the examination can be reduced. Advantageously, the first light cone is dimensioned so that it illuminates the available field of view. In general, a light cone is understood to be an illuminated solid-angle area that has, for example, a conical or pyramidal shape. The light cones could also each be formed by superimposing several elementary, geometric beam shapes.

Advantageously, the optical axes or center axes of the first light cone and the second light cone are oriented parallel to each other, wherein the spacing of the axes is small in comparison with the spacing of the light outlets to the objects of the field of view.

For example, it can be provided that the second light cone is arranged within the first light cone. Here, the relationships in the near field of the light outlets should be disregarded, wherein the near field is small with respect to the distance of the light outlets to the considered objects. Here it is advantageous that the surgeon can already orient the distal end of the endoscope toward the object to be examined before turning on the second light cone, with this object possibly already being dimly recognizable in the first light cone. Here it is especially favorable when the second light cone is centered relative to the first light cone. Thus, the surgeon can orient the endoscope intuitively and focus on the structure of interest. For example, for the centered orientation it can be provided that the first light cone and the second light cone are oriented concentrically.

Various uses are achieved when the endoscope has a flexible construction.

According to one construction it can be provided that the second light outlet is connected to a second optical waveguide that is constructed separately from the first optical waveguide. Here it is advantageous that the activation device does not have to be constructed on the distal end, but instead can be arranged at a distance from this end. This simplifies handling and allows the smallest possible structural dimensions of the distal end.

The light outlets can be supplied from a common light source. However, it is favorable when the second light outlet is connected to a second light source that can be turned on. Here, the second light cone can be turned on in a simple way, in that the second light source is turned on.

A compact construction of the endoscope set is achieved if the first light source and/or the second light source is/are arranged integrated in the endoscope. For example, the light sources could be arranged integrated in a handle of the endoscope.

For simple handling it can be provided that the first optical waveguide and/or the second optical waveguide is/are arranged integrated in a shaft or tube of the endoscope.

It is especially favorable if all of the components of the endoscope set are arranged integrated in the endoscope.

The optical waveguides can be constructed advantageously as quartz fibers or as plastic fibers or as other light-conducting fibers.

It is favorable if the first light outlet and/or the second light outlet is/are constructed as fiber optics. Here it is advantageous that small dimensions of the distal end of the endoscope can be achieved.

For the most uniform illumination possible of the field of view of a wide-angle objective, it is favorable if the first angle of beam spread equals at least 100°, advantageously at least 120°.

For illuminating structures at a farther distance, for example, in ramifications of bronchia, it is favorable if the second angles of beam spread equals at most 40°, advantageously at most 25°. However, other angles of beam spread, in particular, angles of beam spread that are smaller than the specified values, could also be used. Here it is advantageous that the illuminating power of a standard light source is bundled in the smallest possible angle of beam spread, producing an increased range of the light beams.

For example, it could be provided that the second light outlet has an aperture of no more than 0.5, in particular, no more than 0.3 or 0.25 or 0.1.

For one construction of the invention it is provided that the first light source and the second light source are designed for generating light with spectral distributions that are different from each other. For example, the first light source could generate light with a natural spectral distribution or with a spectral distribution suitable for standard examinations and the second light source could emit light of a certain wavelength or in a special spectral range with which certain tissue damage or changes in the tissue could be better identified. It could be provided that the surgeon could influence or specify the spectral range of the second light source.

The activation device can be designed for manual activation.

It can also be provided that the activation device is designed for dimming the first light cone when the second light outlet is turned on. For example, here the first light source could be dimmed. Here it is advantageous that for the examination of details, border areas not of interest could be masked out and thus could be kept from disturbing the surgeon. Turning off or darkening the first light source could even be provided. Advantageously, a mechanical or electrical coupling between the first light source and the second light source could be provided. Dimming or turning off could be performed, for example, mechanically by a moving or adjustable aperture or electronically by controlling the illuminating power generated by the light source.

The activation device can have mechanical switches, for example, moving apertures. It is especially favorable, however, if the activation device has an electrical switch. Here it is advantageous that the fewest possible parts that are subject to mechanical wear and risk of functional deterioration by contamination are used in the endoscope set.

For example, the electrical switch could be constructed as a key switch or pushbutton switch. It could also be provided that the electrical switch is connected to changeover electronics. The changeover electronics can be designed for changing from an operating state in which the second light cone is not turned on into an operating state in which the second light cone is turned on.

For one construction of the invention it can be provided that the second light outlet and/or the second optical waveguide is/are arranged detachably on the endoscope. Here it is advantageous that in this way the components provided for generating the second light cone are arranged so that they can be removed and, after removing these components, an additional lumen or an additional work channel is formed by which additional instruments, laser fibers, mini-endoscopes, or catheters can be provided in a mother-baby technique.

The invention can be used advantageously, in general, in endoscopes that are designed and provided for examining complex, for example, ramified internal spaces.

### BRIEF DESCRIPTION OF THE DRAWINGS

The invention will now be described in detail with reference to an embodiment, but it is not limited to this embodiment. By combining individual or multiple features of the claims with each other or with individual or multiple features of the embodiment, additional embodiments are produced.

Shown in a partially schematic diagram are

FIG. 1 is a side view of an endoscope set according to the invention,

FIG. 2 is a detail view of the handle of the endoscope set from FIG. 1 as a section along C-C,

FIG. 3 is a detail view of the activation device of the endoscope set according to FIG. 1, and

FIG. 4 is a view showing the relative arrangement, according to the invention, of the light cone of the endoscope set relative to each other.

### DETAILED DESCRIPTION OF THE PREFERRED EMBODIMENTS

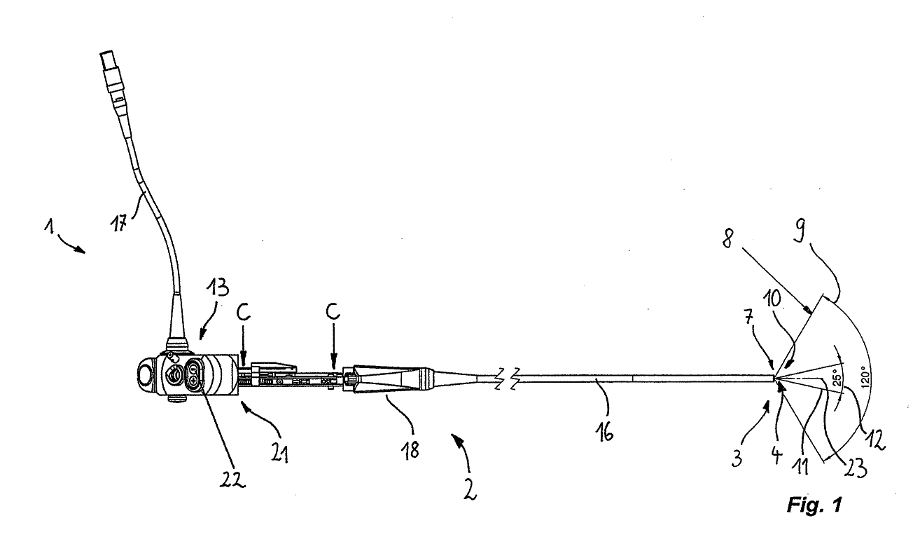

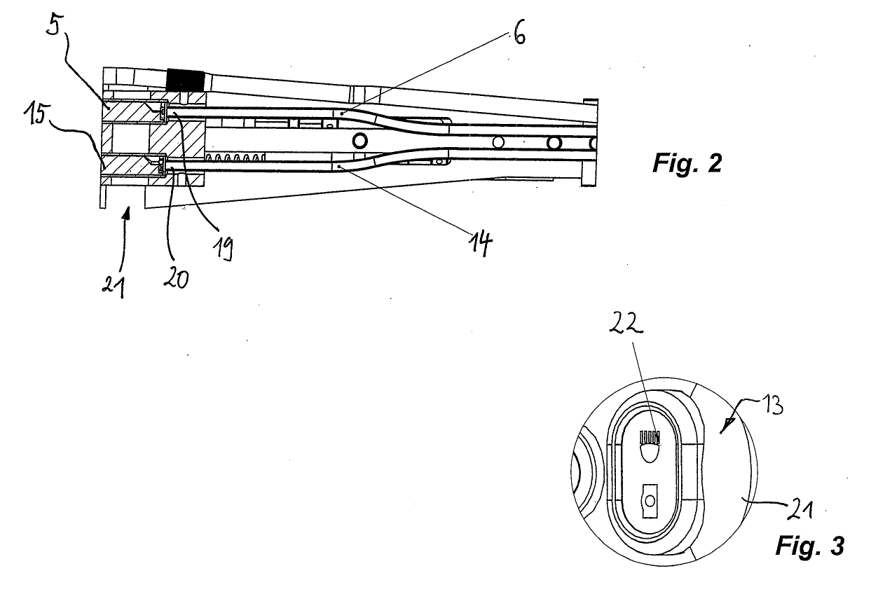

FIG. 1 shows an endoscope set designated as a whole with 1 for bronchoscopy. FIGS. 2 and 3 show different details that are described in common with FIG. 1.

The endoscope set 1 comprises an endoscope 2. For illuminating the field of view of a wide-angle objective 4 that is arranged at the distal end 3 and is not shown further, a first light source 5 is provided, cf. FIG. 2.

The light generated by the first light source 5 is fed via a first optical waveguide 6 to a first light outlet 7 from which the light exits as a first light cone 8 and spreads out with a first angle of beam spread 9 for illuminating the field of view of the wide-angle objective 4 and thus of the endoscope 2.

In the embodiment, an angle of beam spread 9 of 120° is shown, but smaller or larger angles of beam spread are also realized for other embodiments.

At the distal end 3 of the endoscope 2, a second light outlet 10 is further constructed. This second light outlet 10 is designed for illuminating the field of view of the endoscope 2 with light in a second light cone 11 with a second angle of beam spread 12.

The second angle of beam spread 12 is less than the first angle of beam spread 9 of the first light cone 8. Thus, the light emerging from the second light outlet 10 is more strongly bundled than the light emerging from the first light outlet 7.

The first light outlet 7 and second light outlet 10 are constructed in a way that is not shown further as fiber optics so that the described light cones 9, 11 are formed.

In the embodiment, the second angle of beam spread 12 equals 25°, but smaller or larger angles of beam spread could also be realized.

The fiber optics of the second light outlet 10 could be constructed with an aperture of 0.25 or less.

The endoscope 1 has an activation device 13 with which the second light cone 11 can be turned on in addition to the first light cone 8 or instead of the first light cone 8.

For this purpose, the second light outlet 10 is connected via a second optical waveguide 14 to a second light source 15, cf. FIG. 2.

The second light source 15 can be turned on electrically with the activation device 13.

The endoscope 2 has a flexible construction and therefore has an elastic tube 16 in which the image collected by the wide-angle objective 4 is fed to a connection line 17 and from there farther to an eyepiece not shown in more detail or to a camera similarly not shown in more detail.

The elastic tube 16 could be guided and bent with the adjustment device 18.

In the tube 16, the first optical waveguide 6 and the second optical waveguide 14 are also integrated and guided separately from each other to the light outlets 7, 10, respectively, at the distal end 3.

In order to not negatively affect the movement of the flexible tube 16, the optical waveguides 6, 14 are formed from flexible optical fibers.

At their proximal ends 19, 20, the optical waveguides 6, 14 are connected to the first light source 5 or to the second light source 15 that are both arranged integrated in the handle 21 of the endoscope 2.

The activation device 13 has an electrical switch 22 that is constructed as a key switch, cf. FIG. 3.

The switch 22 is connected to changeover electronics that are not shown in more detail and with which the second light cone 11 can be turned on and off, wherein the first light cone 8 is simultaneously dimmed by the changeover electronics in its brightness. The changeover electronics are likewise arranged integrated in the handle 21 of the endoscope 2.

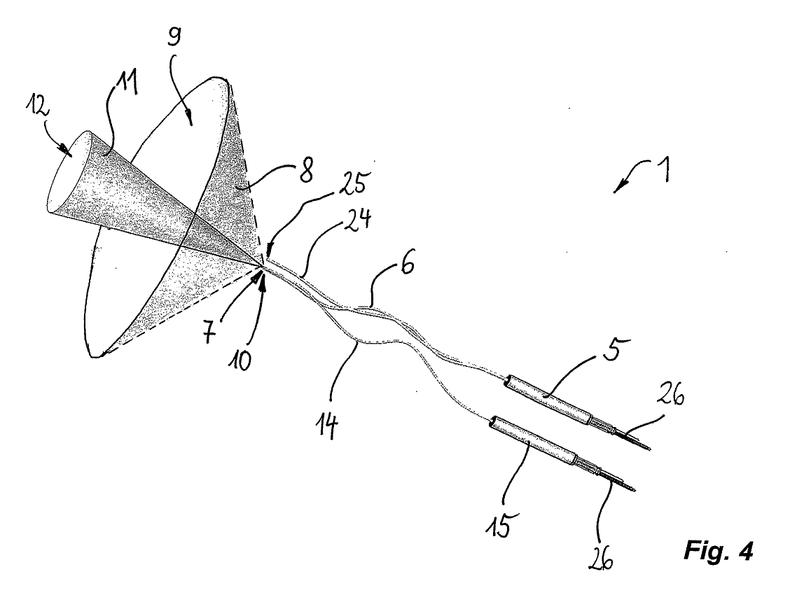

FIG. 4 shows, in a block diagram, the first light cone 8 and the second light cone 11. It is obvious that the second light cone is arranged within the first light cone at least outside of a near field of the light outlets 7, 10.

Through suitable orientation of the light outlets 7, 10, it can be achieved that the optical axes 23 specified by the fiber optics run parallel to each other. In relation to the distances to the objects of interest, the offset of the light outlets 7, 10 is so small relative to each other that the optical axes 23 could be viewed approximately as one optical axis 23, so that the conical light cones 8, 11 lie concentric to each other.

The second light cone 11 is oriented centered to the first light cone 8.

Another optical waveguide 24 is connected to the first light source 5, wherein this optical waveguide has, in the same way as the optical waveguide 6, a light outlet 25 for emitting a light cone that essentially matches the light cone 8. With the activation device 13, this light outlet can be deactivated as shown in FIG. 4 if the second light source 15 is turned on, while the first light outlet 7 continues to be active. In this way, dimming is already realized in the area of the light cone 8.

During operation without light from the second light outlet 10, the light outlets 7 and 25 are turned on by the changeover electronics of the activation device 13, with the beam shapes generated by each of these outlets overlapping for forming a common, first light cone 8.

For controlling the light sources 5, 15 with the activation device 13, these are each connected by connection cables 26. The energy required for operating the light sources 5, 15 and the activation device 13 are fed via the connection line 17.

The second light source 15 is designed for generating light whose spectral distribution is different from the spectral distribution of the light from the first light source 5. Because both light sources 5, 15 are constructed as LEDs, this can be achieved through suitable selection of the LEDs or through preceding color filters.

The second light outlet 10, the second optical waveguide 14, and optionally the second light source 15 could be arranged for one embodiment on the endoscope 2 in the tube 16 so that they can be removed, so that, instead of these parts, an additional lumen could be formed in the tube 16 through which additional instruments, optical fibers, mini-endoscopes, and/or catheters could be guided to the distal end 3 and could be provided there.

For the endoscope set 1, at least two light outlets 7, 10 are provided for illuminating the field of view, with each light outlet forming a light cone 8, 11, wherein the angles of beam spread 9, 12 of the light cones 8, 11 are different sizes and at least the light cone 11 with the smaller angle of beam spread 12 can be turned on.

1. Endoscope set (1) comprising an endoscope (2), at least one first light source (5) and at least one first optical waveguide (6), the first optical waveguide (6) is attached to the first light source (5) and is connected at a distal end (3) of the endoscope (2) to a first light outlet (7), with the first light outlet (7) being designed for illuminating a field of view of the endoscope (2) with light from the first light source (5) in a first light cone (8) with a first angle of beam spread (9), a second light outlet (10) is provided at the distal end (3) of the endoscope (2), with the second light outlet (10) being designed for illuminating the field of view of the endoscope (2) with light in a second light cone (11) with a second angle of beam spread (12), and the second angle of beam spread (12) is smaller than the first angle of beam spread (9), and an activation device (13) is provided to control activation of the second light cone (11).

2. Endoscope set (1) according to claim 1, wherein the second light cone (11) is arranged, at least outside of a near field around the distal end (3), within the first light cone (8).

3. Endoscope set (1) according to claim 1, wherein the second light cone (11) is centered relative to the first light cone (8) or the first light cone (8) and the second light cone (11) are oriented concentric to each other.

4. Endoscope set (1) according to claim 1, wherein the endoscope (2) has a flexible construction.

5. Endoscope set (1) according to claim 1, wherein the second light outlet (10) is connected to a second optical waveguide (14) that is constructed separately from the first optical waveguide (6), or wherein the second light outlet (10) is connected to a second light source (15) that is activatable.

6. Endoscope set (1) according to claim 5, wherein at least one of the first light source (5) and the second light source (15) is arranged integrated in the endoscope (2).

7. Endoscope set (1) according to claim 5, wherein at least one of the first optical waveguide (6) or the second optical waveguide (14) are arranged integrated in a shaft or tube (16) of the endoscope (2).

8. Endoscope set (1) according to claim 1, wherein at least one of the first light outlet (7) or the second light outlet (10) are constructed as optic fibers.

9. Endoscope set (1) according to claim 1, wherein the first angle of beam spread (9) equals at least 100° and the second angle of beam spread (12) equals at most 40°.

10. Endoscope set (1) according to claim 1, wherein the second light outlet (10) has an aperture of no more than 0.5.

11. Endoscope set (1) according to claim 1, wherein the first light source (5) and the second light source (15) are adapted to generate light with spectral distributions that are different from each other.

12. Endoscope set (1) according to claim 1, wherein the activation device (13) is adapted to dim the first light cone (8) when the second light outlet (10) is turned on or the activation device (13) has an electrical switch (22) and is connected to changeover electronics.

13. Endoscope set (1) according to claim 1, wherein at least one of the second light outlet (10) or a second optical waveguide (14) are arranged detachably on the endoscope (2).
